(19) United States
(12) Patent Application Publication
(10) Pub. No.: US 2008/0319068 A1
(43) Pub. Date: Dec. 25, 2008

(54) Novel Polymorph of Acetylsalicylic Acid, and Methods of Making and Using the Same

(75) Inventors: Orn Almarsson, Shrewsbury, MA (US); Jennifer McMahon, Tampa, FL (US); Vishweshwar Peddy, Andhra Pradesh (IN); Matthew Peterson, Hopkinton, MA (US); Michael J. Zaworotko, Tampa, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignees: Transform Pharmaceuticals, Inc., Lexington, MA (US); University of South Florida, Tampa, FL (US)

(21) Appl. No.: 11/909,902

(22) PCT Filed: Apr. 25, 2006
(86) PCT No.: PCT/US06/15767

§ 371 Date: Sep. 8, 2008

Related U.S. Application Data
(60) Provisional application No. 60/675,262, filed on Apr. 27, 2005.

(51) Int. Cl. A61K 31/216; C07C 69/66; A61P 25/04

(52) U.S. Cl. 514/548; 560/193

(57) ABSTRACT

A polymer of aspirin is provided by the present invention. Methods of making and using the same are also provided.

### FIELD OF THE INVENTION

The present invention relates to acetylsalicylic acid, pharmaceutical compositions comprising acetylsalicylic acid, and methods for preparing and using the same.

### BACKGROUND OF THE INVENTION

Many drugs in pharmaceutical compositions can be prepared in a variety of different forms. Such drugs can be prepared so as to have a variety of different chemical forms including chemical derivatives or salts. Such drugs can also be prepared to have different physical forms. For example, the drugs may be amorphous or may have different crystalline polymorphs. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

It would be advantageous to have a new form of acetylsalicylic acid that has improved properties, in particular, as an oral formulation. Specifically, it is desirable to identify improved forms of the drug that exhibit significantly increased aqueous solubilities, stability, and/or hygroscopicity. It is also desirable to increase the dissolution rate of drug-containing pharmaceutical compositions in water, increase the bioavailability of orally-administered compositions, and provide a more rapid onset to therapeutic effect. It is also desirable to have a form of the drug which, when administered to a subject, reaches a peak plasma level faster and/or has a longer lasting plasma concentration and higher overall exposure at high doses when compared to equivalent amounts of the drug in its presently-known form.

Acetylsalicylic acid, also known as aspirin, has the structure of Formula A:

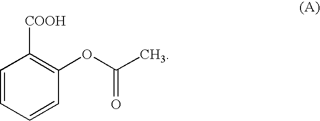

### SUMMARY OF THE INVENTION

The invention provides a novel polymorph of acetylsalicylic acid. The invention also provides novel pharmaceutical compositions comprising this novel form and related methods of treatment.

Compositions of the invention are useful as an analgesic or an anti-inflammatory, for example. The compositions and methods of the invention are useful for treating one or more of the following: pain, such as a headache or arthritis pain, fever, pre-eclampsia, heart attack and predisposition of heart attack. In one embodiment, a method comprises administering to a mammal a therapeutically-effective amount of acetylsalicylic acid Form II.

In another aspect, the present invention provides a method of making acetylsalicylic acid Form II, which comprises:
(a) mixing acetylsalicylic acid, with levetiracetam and an appropriate solvent; and
(b) crystallizing the acetylsalicylic acid, under conditions which lead to the formation of Form II.

In another embodiment, the use of acetylsalicylic acid Form II described herein can be used in the preparation of a medicament for treating a mammal in need of such treatment. In another embodiment, the invention also provides a novel medicament comprising acetylsalicylic acid Form II as described in the present application and related methods of treatment.

These and other embodiments of the invention are described further in the detailed description of the invention.

### BRIEF DESCRIPTION OF THE DRAWINGS

FIG. 1—IR spectra of acetylsalicylic acid Form II (top spectrum) and Form I (bottom spectrum)

FIG. 2—DSC thermogram of acetylsalicylic acid Form II

FIG. 3—USC thermogram overlay of acetylsalicylic acid Form II (dashed line) and Form I (solid line)

### DETAILED DESCRIPTION OF THE INVENTION

The invention provides a novel polymorph of acetylsalicylic acid. The invention also provides novel pharmaceutical compositions comprising this novel form and related methods of treatment.

In one embodiment, the present invention is directed to acetylsalicylic acid Form II.

In another embodiment, the present invention is directed to a method of making acetylsalicylic acid Form II, comprising:
(a) mixing acetylsalicylic acid, with levetiracetam and an appropriate solvent; and
(b) crystallizing the acetylsalicylic acid, under conditions which lead to the formation of Form II.

Compositions of the invention are useful as an analgesic or an anti-inflammatory, for example. The compositions and methods of the invention are useful for treating one or more of the following: pain, such as a headache or arthritis pain, fever, pre-eclampsia, heart attack and predisposition of heart attack. In another embodiment, a method is provided which comprises administering to a mammal a therapeutically-effective amount of acetylsalicylic acid Form II.

In another embodiment, the use of acetylsalicylic acid Form II described herein can be used in the preparation of a medicament for treating a mammal in need of such treatment. In another embodiment, the invention also provides a novel medicament comprising acetylsalicylic acid Form II as described in the present application and related methods of treatment.

Pharmaceutical dosage forms of acetylsalicylic acid Form II can be administered orally, parenterally, by inhalation spray, topically, rectally, nasally, buccally, vaginally or via an implanted reservoir. Oral and parenteral pharmaceutical compositions and dosage forms are exemplary dosage forms. Optionally, the oral dosage form is a solid dosage form, such as a tablet, a caplet, a hard gelatin capsule, a starch capsule, a hydroxypropyl methylcellulose (HPMC) capsule, or a soft elastic gelatin capsule. Other dosage forms include an intradermal dosage form, an intramuscular dosage form, a subcutaneous dosage form, and an intravenous dosage form.

Acetylsalicylic acid Form II can be administered by controlled- or delayed-release means. Controlled-release pharmaceutical products generally have a common goal of improving drug therapy over that achieved by their non-controlled release counterparts. Ideally, the use of an optimally designed controlled-release preparation in medical treatment is characterized by a minimum of API substance being employed to cure or control the condition in a minimum amount of time. Advantages of controlled-release formulations generally include: 1) extended activity of the API; 2) reduced dosage frequency; 3) increased patient compliance; 4) usage of less total API; 5) reduction in local or systemic side effects; 6) minimization of API accumulation; 7) reduction in blood level fluctuations; 8) improvement in efficacy of treatment; 9) reduction of potentiation or loss of API activity; and 10) improvement in speed of control of diseases or conditions. (Kim, Cherng-ju, Controlled Release Dosage Form Design, 2 Technomic Publishing, Lancaster, Pa.: 2000). Topical dosage forms of the invention include, but are not limited to, creams, lotions, ointments, gels, shampoos, sprays, aerosols, solutions, emulsions, and other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity optionally greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, optionally in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18th ed., Mack Publishing, Easton, Pa. (1990).

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are optionally sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Transdermal and mucosal dosage forms of the invention include, but are not limited to, ophthalmic solutions, patches, sprays, aerosols, creams, lotions, suppositories, ointments, gels, solutions, emulsions, suspensions, or other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). Dosage forms suitable for treating mucosal tissues within the oral cavity can be formulated as mouthwashes, as oral gels, or as buccal patches. Further, transdermal dosage forms include “reservoir type” or “matrix type” patches, which can be applied to the skin and worn for a specific period of time to permit the penetration of a desired amount of active ingredient.

Like the amounts and types of excipients, the amounts and specific type of active ingredient in a dosage form may differ depending on factors such as, but not limited to, the route by which it is to be administered to patients. However, typical daily dosage forms of the invention comprise acetylsalicylic acid Form II in an amount of from about 1 mg to about 5000 mg, from about 50 mg to 2500 mg, or from about 100 mg to about 1000 mg.

In one embodiment of the invention, a pharmaceutical composition comprising acetylsalicylic acid Form II is administered orally as needed in an amount of from about 50 mg to about 1000 mg, from about 50 mg to about 750 mg, or from about 50 mg to about 500 mg. In specific embodiments, pharmaceutical compositions comprising a acetylsalicylic acid form of the present invention can be administered orally in amounts of about 81, 325, or 500 mg. The dosage amounts can be administered in single or divided doses.

In other embodiments, the present invention is directed to compositions comprising acetylsalicylic acid Form II as described herein and one or more diluents, carriers, and/or excipients suitable for the administration to a mammal for the treatment or prevention of one or more of the conditions described herein.

Typical pharmaceutical compositions and dosage forms comprise one or more excipients. Suitable excipients are well known to those skilled in the art of pharmacy, and non-limiting examples of suitable excipients are provided herein. For example, excipients that can be used in oral dosage forms of the invention include, but are not limited to, binders, stabilizers, fillers, disintegrants, and lubricants. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. In addition, pharmaceutical compositions or dosage forms may contain one or more compounds that reduce or alter the rate by which the active ingredient will decompose. Such compounds, which are referred to herein as “stabilizers”, include, but are not limited to, antioxidants, pH buffers, or salt buffers.

Acetylsalicylic acid can be made using various methods known to those skilled in the art. For example, U.S. Pat. Nos. 2,890,240 and 3,235,583 disclose acetylsalicylic acid and methods of preparing it. Of course, other methods known to one of ordinary skill in the art may be used to prepare acetylsalicylic acid.

The invention is described further in the following example, which is illustrative and in no way limiting.

Uses for acetylsalicylic acid are well known in the art and include the treatment of pain such as headache and arthritis pain, fever, pre-eclampsia, heart attack and predisposition of heart attack. The dosage and administration for acetylsalicylic acid compositions of the present invention can be determined using routine methods in the art but will generally be about those dosages recommended by the package inserts (or Physician's Desk Reference) for acetylsalicylic acid.

The previously known form of acetylsalicylic acid, herein referred to as acetylsalicylic acid, Form I, or acetylsalicylic acid Form I, is commercially available by Bayer AG.

### EXAMPLE

### Analytical Methods

Differential scanning calorimetric (DSC) analysis of the samples was performed using a Q1000 Differential Scanning Calorimeter (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/95/2000NT, version 3.1E; Build 3.1.0.40 (2001 TA Instruments-Water LLC).

For the DSC analysis, the purge gas used was dry nitrogen, the reference material was an empty aluminum pan that was crimped, and the sample purge was 50 mL/minute.

DSC analysis of the samples were performed by placing the acetylsalicylic acid sample in an aluminum pan with a crimped pan closure. The starting temperature was typically 20 degrees C. with a heating rate of 10 degrees C./minute, and the ending temperature was 250 degrees C.

Single crystal x-ray crystallographic analyses conducted in connection with the experiments described herein were used to determine unit cell dimensions, space group, and atomic position of all atoms in a compound relative to the origin of its unit cell. The unit cell dimension is defined by three parameters; length of the sides of the cell, relative angles of sides to each other and the volume of the cell. The lengths of the sides of the unit cell are defined by a, b and c. The relative angles of the cell sides are defined by alpha, beta, and gamma. The volume of the cell is defined as V. A more detailed account of unit cells can be found in Chapter 3 of Stout & Jensen, X-Ray Structure Determination; A Practical Guide, Mac Millian Co., New York, N.Y. (1968).

The results of a single crystal x-ray analysis are limited to the crystal placed in the x-ray beam. Crystallographic data on a large group of crystals provides powder x-ray diffraction. If the powder is a pure crystalline compound a simple powder diagram is obtained. To compare the results of a single crystal analysis and powder x-ray analysis a simple calculation can be done converting the single crystal data into a powder x-ray diagram, SHELXTL Plus® computer program, Reference Manual by Siemens Analytical X-ray Instrument, Chapter 10, p. 179-181, 1990. This conversion is possible because the single crystal experiment routinely determines the unit cell dimensions, space group, and atomic positions. These parameters provide a basis to calculate a perfect powder pattern. Comparing this calculated powder pattern and the powder pattern experimentally obtained from a large collection of crystals will confirm if the results of the two techniques are the same.

Single crystal x-ray data were collected on a Bruker SMART-APEX CCD diffractometer (M. J. Zaworotko, Department of Chemistry, University of South Florida). Lattice parameters were determined from least squares analysis. Reflection data was integrated using the program SAINT. The structure was solved by direct methods and refined by full matrix least squares using the program SHELXTL (Sheldrick, G. M. SHELXTL, Release 5.03; Siemans Analytical X-ray Instruments Inc.: Madison, Wis.).

Any one, two, three, four, five, or six DSC transitions can be used to characterize the compositions of the present invention. Single-crystal data and melting points can also be used separately, or together to characterize a composition of the present invention.

### Example 1

### Acetylsalicylic Acid Form II

To acetylsalicylic acid (16 mg) was added levetiracetam (15 mg). To the solid mixture was added acetonitrile (1 mL) and the solution was heated at 70 degrees C. for 5 minutes. The homogeneous solution was then slowly evaporated for 2 days. After 2 days, a precipitate was observed, collected, and dried to give acetylsalicylic acid Form II as small colorless plates. The crystals were characterized using IR, DSC, melting point, and single-crystal x-ray analysis.

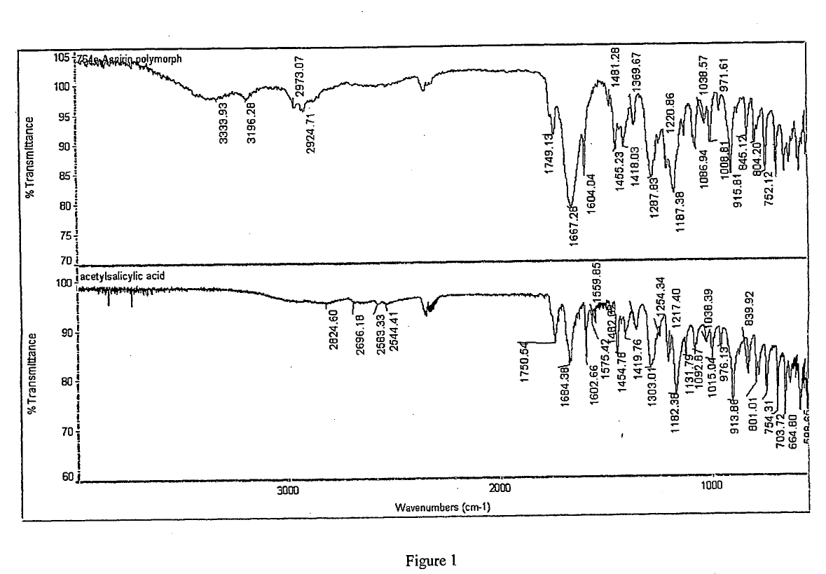

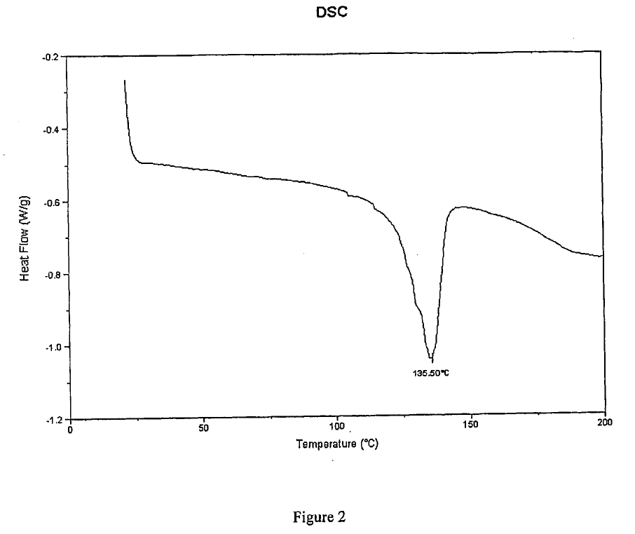

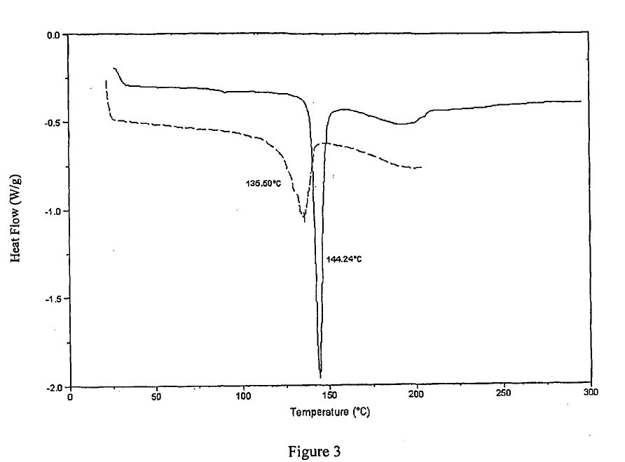

The acetylsalicylic acid Form II can be characterized by any one, any two, any three, any four, any five, or any six or more of the IR peaks in FIG. 1 (top spectrum) including, but not limited to, 1749, 1667, 1604, 1455, 1418, 1288, 1187, 1087, 1009, 916, 845, 804, and 752 cm⁻¹. The bottom IR spectrum in FIG. 1 shows data for acetylsalicylic acid Form I. The DSC thermogram shows an endothermic transition at about 135.5 degrees C. (FIG. 2). FIG. 3 shows a comparison of DSC thermograms for acetylsalicylic acid, Forms I and II (solid line=Form I, dashed line Form II). The melting point of acetylsalicylic acid Form II was determined to be about 128-130 degrees C., using a mel-temp apparatus.

Single-crystal x-ray data: C₉H₈O₄, M=180.16, monoclinic P2(1)/c; a=12.095(7) Å, b=6.491(4) Å, c=11.323(6) Å, alpha=90°, beta=111.509(9)°, gamma=90°, T=100(2) K, Z=4, D_{c}=1.447 g/cm³, V=827.1(8) Å³, wavelength 0.71073 Å.

1. Acetylsalicylic acid Form II, wherein said acetylsalicylic acid Form II exhibits crystal parameters that are approximately equal to the following:
Monoclinic, P2₁/c; a=12.095(7) Å, b=6.491(4) Å, c=11.323(6) Å; β=111.509(9)°: V=827.1(8) Å³; Z=4.

2. (canceled)

3. The acetylsalicylic acid Form II of claim 1, wherein said acetylsalicylic acid Form II exhibits a melting point at about 128-130 degrees C.

4. The acetylsalicylic acid Form II of claim 1, wherein said acetylsalicylic acid Form II exhibits an IR spectrum comprising peaks selected from the group consisting of:
(i) 1667, 1455, 1288, and 1187 cm⁻¹;
(ii) 1087, 1009, 916, and 752 cm⁻¹; and
(iii) 1667, 1288, 1009, and 916 cm⁻¹.

5. A method of making acetylsalicylic acid Form II, comprising:
(a) mixing acetylsalicylic acid with levetiracetam and an appropriate solvent; and
(b) crystallizing acetylsalicylic acid under conditions which lead to the formation of Form II according to claim 1.

6. A pharmaceutical dosage form comprising a therapeutically effective amount of said acetylsalicylic acid Form II of claim 1.

7. A pharmaceutical dosage form comprising a pharmaceutically acceptable carrier, diluent, or excipient and a therapeutically effective amount of said acetylsalicylic acid Form II of claim 1.

8. A method of treating a mammal with a condition selected from the group consisting of: pain, headache pain, arthritis pain, fever, pre-eclampsia, heart attack, and predisposition of heart attack, which comprises administering to the mammal a therapeutically effective amount of acetylsalicylic acid Form II according to claim 1.
